(19) United States
(12) Patent Application Publication
(10) Pub. No.: US 2022/0298573 A1
(43) Pub. Date: Sep. 22, 2022

(54) DETERMINATION OF AN INFECTION PROFILE IN AN INDIVIDUAL AND USES THEREOF

(71) Applicant: Curative Inc., San Dimas, CA (US)

(72) Inventors: Frederick Edward TURNER, San Jose, CA (US); Vladimir I. SLEPNEV, Diamond Bar, CA (US)

(21) Appl. No.: 17/617,899

(22) PCT Filed: Jun. 10, 2020
(86) PCT No.: PCT/US2020/037040

§ 371 Date: Dec. 9, 2021

Related U.S. Application Data
(60) Provisional application No. 62/859,616, filed on Jun. 10, 2019.

(51) Int. Cl. C12Q 1/6883

(52) U.S. Cl. CPC C12Q 1/6883; C12Q 2600/158

(57) ABSTRACT

The present disclosure, in some aspects, is directed to methods of determining an infection profile of an individual, wherein the infection profile is based on a host response profile and a pathogen profile of the individual. In other aspects, the present disclosure is directed to uses (e.g., methods of treatment, treatment selection, patient selection), kits, and compositions of the methods described herein.

### CROSS-REFERENCE TO RELATED APPLICATIONS

This application claims priority benefit to U.S. Provisional Patent Application No. 62/859,616, filed on Jun. 10, 2019, the disclosure of which is hereby incorporated herein by reference in its entirety.

### TECHNICAL FIELD

The present disclosure, in some aspects, is directed to methods of determining an infection profile of an individual, wherein the infection profile is based on a host response profile and a pathogen profile of the individual. In other aspects, the present disclosure is directed to uses (e.g., methods of treatment, treatment selection, patient selection), kits, and compositions of the methods described herein.

### BACKGROUND

Proper and timely diagnosis of pathogen infection is of critical importance for optimal patient care. Developing techniques for reliable diagnosis of pathogen infection on a clinically relevant time scale is challenging, and there currently is a lack of availability of such tests in the clinic. For example, sepsis, a dysregulated host response to bacterial, fungal, or viral infections, is challenging to detect early and accurately, leading to treatment failures and high mortality. Globally, an estimated 30 million patients are affected every year leading to 6 million deaths. In the US, it is the sixth most common reason for hospitalization and is the most expensive condition treated in U.S. hospitals, with an aggregate cost of US$15.4 billion in 2009. Nonspecific diagnosis of sepsis accounts for another US$23.7 billion each year.

Early and accurate determination of bacterial disease etiology is crucial for implementing effective pathogen-targeted therapies but is often not possible due to the limitations of current microbiological tests in terms of sensitivity and turnaround time. The gold standard of current sepsis diagnostics, bacterial culture, typically requires 24-72 hours for detection and identification and additional time for determination of antimicrobial sensitivity testing. The gold standard blood culture is particularly inadequate in the neonatal population where the symptoms often overlap with other non-infectious conditions such as perinatal asphyxia or respiratory distress syndrome.

In the absence of a definitive microbiologic diagnosis, clinicians cannot determine if symptoms are due to a pathogen infection or a non-infectious inflammatory condition. In absence of relevant evidence, or even with negative microbiological testing, clinicians often continue empiric antibiotic treatment due to concerns of not timely treating a bacterial infection, a practice that drives emergence of antibiotic resistance and increases the risk of, e.g., Clostridium difficile infection.

All references cited herein, including patent applications and publications, are incorporated by reference in their entirety.

### BRIEF SUMMARY

In one aspect, the present disclosure provides methods of determining an infection profile of an individual, wherein the infection profile is based on a host response profile and a pathogen profile of the individual, the method comprising: (a) enriching host nucleic acids and pathogen nucleic acids, if present, from a sample from the individual; and (b) determining the host response profile and the pathogen profile to determine the infection profile of the individual.

In some embodiments, the host response profile is indicative of a pathogen infection in the individual. In some embodiments, the pathogen infection is a bacterial infection. In some embodiments, the pathogen infection is a non-bacterial infection. In some embodiments, the host response profile is indicative of an infection-negative response in the individual. In some embodiments, the infection-negative response is infection-negative systemic inflammation.

In some embodiments, the host response profile is based on host nucleic acid levels of one or more host response biomarkers. In some embodiments, the one or more host response biomarkers are selected from any of EACAM4, LAMP1, PLA2G7, PLAC8, or FAIM3. In some embodiments, the host response profile comprises a host response score, such as an index, based on the one or more host response biomarkers.

In some embodiments, the pathogen profile is indicative of the presence of a pathogen, or portion thereof, in the sample from the individual. In some embodiments, the pathogen profile is based on the presence of pathogen nucleic acids. In some embodiments, the pathogen nucleic acids comprise 16S and/or 23S nucleic acids. In some embodiments, the 16S and/or 23S nucleic acids comprise conserved sequences of ribosomal RNA (rRNA) of 16S and/or 23S. In some embodiments, the pathogen profile comprises the identification of the pathogen.

In some embodiments, the pathogen profile comprises an attribute of the pathogen. In some embodiments, the attribute of the pathogen is anti-microbial resistance. In some embodiments, anti-microbial resistance of the pathogen is based on one or more anti-microbial resistance biomarkers. In some embodiments, the one or more anti-microbial resistance biomarkers are indicative of presence of a carbapenemase, presence of an extended spectrum beta-lactamase, methicillin resistance, or vancomycin resistance.

In some embodiments, the infection profile comprises: (i) a host response profile indicative of a pathogen infection in the individual; and (ii) a pathogen profile indicative of the presence of a bacterial pathogen in the individual.

In some embodiments, the infection profile comprises: (i) a host response profile indicative a pathogen infection in the individual; and (ii) a pathogen profile indicative of the presence of a non-bacterial pathogen in the individual. In some embodiments, the non-bacterial pathogen is a fungus or virus.

In some embodiments, the infection profile comprises: (i) a host response profile indicative of an infection-negative response in the individual; and (ii) a pathogen profile indicative of the presence of a bacterial pathogen in the individual.

In some embodiments, the infection profile comprises: (i) a host response profile indicative of an infection-negative response in the individual; and (ii) a pathogen profile indicative of the presence of a non-bacterial pathogen in the individual. In some embodiments, the non-bacterial pathogen is a fungus or virus.

In some embodiments, enriching host nucleic acids and pathogen nucleic acids, if present, comprises subjecting the sample to an oligonucleotide-based affinity matrix. In some embodiments, the oligonucleotide-based affinity matrix is contained in a microfluidic flow cell. In some embodiments, subjecting the sample to the oligonucleotide-based affinity matrix comprises at least 2 cycles of subjecting the sample to the oligonucleotide-based affinity matrix.

In some embodiments, enriching host nucleic acids and pathogen nucleic acids, if present, further comprises eluting captured nucleic acids from the oligonucleotide-based affinity matrix. In some embodiments, eluting captured nucleic acids from the oligonucleotide-based affinity matrix occurs prior to determining the host response profile and the pathogen profile. In some embodiments, nucleic acids are eluted using an elution buffer, and wherein the elution buffer has a pH of about 3 or less, or the elution buffer has a pH of about 12 or greater. In some embodiments, the pH of the elution buffer is neutralized following elution. In some embodiments, the volume of the eluate is less than about 100 μL.

In some embodiments, determining the host response profile comprises detecting the enriched host nucleic acids. In some embodiments, detecting the enriched host nucleic acids comprises subjecting the enriched host nucleic acids to an amplification technique. In some embodiments, determining the pathogen profile comprises detecting the enriched pathogen nucleic acids. In some embodiments, detecting the enriched pathogen nucleic acids comprises subjecting the enriched pathogen nucleic acids to an amplification technique. In some embodiments, the amplification technique is a PCR technique. In some embodiments, the PCR technique comprises a reverse transcription PCR (RT-PCR) technique and/or a quantification PCR (qPCR) technique.

In some embodiments, determining the pathogen profile comprises determining the identification of a pathogen. In some embodiments, determining the identification of the pathogen comprises subjecting the amplified pathogen nucleic acids to a nucleic acid sequencing technique. In some embodiments, the nucleic acid sequencing technique is a nanopore-based sequencing technique.

In some embodiments, the host nucleic acids are RNA. In some embodiments, the host nucleic acids are mRNA.

In some embodiments, the pathogen nucleic acids comprise nucleic acids from one or more pathogens. In some embodiments, the pathogen nucleic acids comprise nucleic acids from a plurality of pathogens, wherein the plurality of pathogens comprises two or more species of pathogens. In some embodiments, the pathogen nucleic acids are RNA. In some embodiments, the pathogen nucleic acids are rRNA.

In some embodiments, the sample is a whole blood sample. In some embodiments, the volume of the whole blood sample is about 1 ml to about 15 ml. In some embodiments, the method further comprises processing the sample prior to the step of enriching. In some embodiments, the method is completed in 24 hours or less.

In another aspect, the present disclosure provides methods of diagnosing sepsis in an individual comprising performing the methods described herein.

In another aspect, the present disclosure provides methods of diagnosing an infectious disease in a patient potentially infected with a pathogenic microorganism, wherein the diagnostic result is based on a host response profile and detection of nucleic acids derived from a pathogenic microorganism in the individual, the method comprising: (a) enriching host nucleic acids and pathogen nucleic acids, if present, from a sample from the individual; (b) determining the host response profile and presence or absence of nucleic acids derived from pathogenic microorganisms; and (c) assessing likelihood of the infectious disease potentially caused by pathogenic microorganism based on interpretation of the host response profile and detection of nucleic acids derived from the pathogenic microorganism. In some embodiments, the host response profile is indicative of a pathogen infection in the individual. In some embodiments, the host response profile is indicative of an infection-negative response in the individual. In some embodiments, the detection of nucleic acids derived from pathogenic microorganism is indicative of the presence of a pathogen, or portion thereof, in the sample from the individual. In some embodiments, detection of nucleic acids derived from pathogenic microorganism comprises detection of nucleic acids encoding 16S ribosomal RNA and/or 23S ribosomal RNA. In some embodiments, detection of nucleic acids derived from the pathogenic microorganism comprises the identification of the pathogen. In some embodiments, the pathogen profile comprises an attribute of the pathogen. In some embodiments, the attribute of the pathogen is anti-microbial resistance. In some embodiments, enriching host nucleic acids and pathogen nucleic acids, if present, comprises subjecting the sample to an oligonucleotide-based affinity matrix. In some embodiments, the oligonucleotide-based affinity matrix is contained in a microfluidic flow cell. In some embodiments, determining the host response profile comprises detecting the enriched host nucleic acids. In some embodiments, determining presence of nucleic acids derived from a pathogenic microorganism comprises determining the identification of a pathogen.

In another aspect, the present disclosure provides methods of differentiating a disease-causing infection in a patient potentially infected with a pathogenic microorganism from a non-disease causing infection or specimen contamination by a microorganism, wherein the diagnostic result is based on a host response profile and on detection of nucleic acids derived from a pathogenic microorganism of the individual, the method comprising: (a) enriching host nucleic acids and pathogen nucleic acids, if present, from a sample from the individual; and (b) determining the host response profile and presence or absence of nucleic acids derived from pathogenic microorganisms; and (c) assessing likelihood of infectious disease potentially caused by pathogenic microorganism based on interpretation of the host response profile and detection of nucleic acids derived from the pathogenic microorganism.

These and other aspects and advantages of the present disclosure will become apparent from the subsequent detailed description and the appended claims. It is to be understood that one, some, or all of the properties of the various embodiments described herein may be combined to form other embodiments of the present disclosure.

### DETAILED DESCRIPTION

The present disclosure is based, in part, on the inventor's unique insight that techniques for measurements of host response and detection of a pathogen infection (or lack thereof) can be integrated and improved to provide, in clinically relevant time frame, information necessary to make clinically appropriate decisions regarding patient treatment thus allowing for improve patient outcomes.

In some aspects, the present disclosure describes methods of determining an infection profile of an individual, wherein the infection profile is based on a host response profile and a pathogen profile of the individual. In some embodiments of the methods described herein, the host response profile is indicative of a pathogen infection in the individual. In some embodiments of the methods described herein, the host response profile is indicative of an infection-negative response in the individual. In some embodiments, the pathogen profile is indicative of the presence (including the lack thereof) of a pathogen, or portion thereof, (such as a bacterium) in the sample from the individual. These measurements, when integrated, can provide, e.g., an indication of positive bacterial detection and strong host response, which will positively diagnose bacterial sepsis and support the need for antibiotic therapy. Identification of the bacterial pathogen, and one or more specific attributes thereof, will provide further guidance on treatment selection. Other infection profiles obtained from the methods described herein may indicate, e.g., the absence of bacterial detection and a low score of a biomarker expression index for host response, which will rule out a bacterial etiology of sepsis and thus negate the need for antibiotic therapy. A low score of a biomarker expression index for host response will help to distinguish asymptomatic or transient bacteremia from sepsis-causing bacteremia, particularly for the detection of commensal or opportunistic microorganisms. The methods described herein are particularly useful as they can be completed within a short time frame following sample acquisition (e.g., 0.5-6 hours), thus allowing timely selection of a clinical approach and patient treatment.

In some aspects, the methods described herein are useful for a specific use, such as a method of treatment. In some embodiments, the methods provided herein provide a molecular testing tool to predict the host response in sepsis and will provide a personalized therapeutic approach to treatment of sepsis by allowing for differential diagnosis of sepsis from non-septic acute infections. In addition to the management of sepsis, the methods described herein can be broadly applied to diagnosis and treatment of other infectious diseases.

Also contemplated herein are the components and kits of the methods disclosed herein.

It will also be understood by those of ordinary skill in the art that changes in the form and details of the implementations described herein may be made without departing from the scope of this disclosure. In addition, although various advantages, aspects, and objects have been described with reference to various implementations, the scope of this disclosure should not be limited by reference to such advantages, aspects, and objects.

### Definitions

As used herein, “treatment” or “treating” is an approach for obtaining beneficial or desired results, including clinical results. For example, for purposes of this application, beneficial or desired clinical results include, but are not limited to, one or more of the following: alleviating one or more symptoms resulting from the disease or the state of the individual, diminishing the extent of the disease or the state of the individual, stabilizing the disease or the state of the individual (e.g., preventing additional weight gain), ameliorating the disease state or the state of the individual, decreasing the dose of one or more other medications required to treat the disease or the state of the individual, increasing or improving the quality of life, decreasing weight gain, and/or increasing weight loss. The methods of the invention contemplate any one or more of these aspects of treatment.

The term “individual” refers to a mammal and includes, but is not limited to, human, bovine, horse, feline, canine, rodent, or primate.

The terms “comprising,” “having,” “containing,” and “including,” and other similar forms, and grammatical equivalents thereof, as used herein, are intended to be equivalent in meaning and to be open ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

Where a range of values is provided, it is understood that each intervening value, to the tenth of the unit of the lower limit, unless the context clearly dictate otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

Reference to “about” a value or parameter herein includes (and describes) variations that are directed to that value or parameter per se. For example, description referring to “about X” includes description of “X.”

As used herein, including in the appended claims, the singular forms “a,” “or,” and “the” include plural referents unless the context clearly dictates otherwise.

Those skilled in the art will recognize that several embodiments are possible within the scope and spirit of the present disclosure. The following description illustrates the disclosure and, of course, should not be construed in any way as limiting the scope of the inventions described herein.

In some aspects, provided herein are methods of determining an infection profile of an individual, wherein the infection profile is based on a host response profile and a pathogen profile of the individual. In some embodiments, the term profile, such as used in reference to an infection profile, a host response profile, and a pathogen profile, refers to one or more information/data points associated with the profile. In some embodiments, the data of a profile may include different types of information, e.g., index scores, expression levels, presence versus absence, identification information, and attribute information. In some embodiments, the infection profile is indicative of an infection-positive response in the individual. In some embodiments, the host response profile is indicative of an infection-negative response in the individual. In some embodiments, the host response profile is indicative of the likelihood of the infectious disease potentially caused by pathogenic microorganism in the individual. In some embodiments, the pathogen profile is indicative of a bacterial infection, e.g., sepsis. In some embodiments, the pathogen profile is indicative of a non-bacterial infection or condition.

In some embodiments, the method of determining an infection profile of an individual, wherein the infection profile is based on a host response profile and a pathogen profile of the individual, the method comprising: (a) enriching host nucleic acids and pathogen nucleic acids, if present, from a sample from the individual; and (b) determining the host response profile and the pathogen profile to determine the infection profile of the individual.

In some embodiments, the host response profile is indicative of a pathogen infection in the individual, e.g., the absence or presence of a pathogen infection in the individual. In some embodiments, the pathogen infection is a bacterial infection. In some embodiments, the pathogen infection is a non-bacterial infection, such as an infection by a fungus, parasite, or virus.

In some embodiments, the host response profile is indicative of an infection-negative response in the individual. In some embodiments, the infection-negative response is infection-negative systemic inflammation.

In some embodiments, the host response profile is based on host nucleic acid levels of one or more host response biomarkers. In some embodiments, one or more host response biomarkers are selected from any of EACAM4, LAMP1, PLA2G7, PLAC8, or FAIM3. In some embodiments, the host response profile comprises a host response score based on the one or more host response biomarkers.

In some embodiments, the host response profile is based on host cell polypeptides, e.g., host cell proteins. In some embodiments, the host response profile is based on expression level or composition of a host cell protein, e.g., overexpression of a host cell protein, a post-translation modification of a host cell protein.

Methods for determining a host response profile, and/or information contained therein, are known in the art. See, e.g., Maslove et al., Journal of Critical Care, 49 (2019), which is hereby incorporated by reference in its entirety.

In some embodiments, the pathogen profile is indicative of the presence of a pathogen, or portion thereof, in the sample from the individual, e.g., the absence or presence of the pathogen or a portion thereof. In some embodiments, the pathogen profile is based on the presence of pathogen nucleic acids. In some embodiments, the pathogen nucleic acids comprise 16S and/or 23S nucleic acids. In some embodiments, the 16S and/or 23S nucleic acids comprise conserved sequences of ribosomal RNA (rRNA) of 16S and/or 23S. In some embodiments, the conserved sequences of rRNA of 16S and/or 23S are conserved across a plurality of pathogens.

In some embodiments, the pathogen profile comprises the identification of the pathogen. In some embodiments, the pathogen profile comprises an attribute of the pathogen. In some embodiments, the attribute of the pathogen is anti-microbial resistance. In some embodiments, anti-microbial resistance of the pathogen is based on one or more anti-microbial resistance biomarkers, e.g., expression or lack thereof a protein involved with anti-microbial resistance. In some embodiments, the one or more anti-microbial resistance biomarkers are indicative of presence of a carbapenemase, presence of an extended spectrum beta-lactamase, methicillin resistance, or vancomycin resistance.

In some embodiments, the infection profile, as determined according to the methods described herein, comprises: (i) a host response profile indicative of a pathogen infection in the individual; and (ii) a pathogen profile indicative of the presence of a bacterial pathogen in the individual.

In some embodiments, the infection profile, as determined according to the methods described herein, comprises: (i) a host response profile indicative a pathogen infection in the individual; and (ii) a pathogen profile indicative of the presence of a non-bacterial pathogen in the individual. In some embodiments, the non-bacterial pathogen is a fungus, parasite, or virus.

In some embodiments, the infection profile, as determined according to the methods described herein, comprises: (i) a host response profile indicative of an infection-negative response in the individual; and (ii) a pathogen profile indicative of the presence of a bacterial pathogen in the individual.

In some embodiments, the infection profile, as determined according to the methods described herein, comprises: (i) a host response profile indicative of an infection-negative response in the individual; and (ii) a pathogen profile indicative of the presence of a non-bacterial pathogen in the individual. In some embodiments, the non-bacterial pathogen is a fungus, parasite or virus.

The methods described herein comprise enriching nucleic acids in a sample from an individual. In some embodiments, the methods described herein comprise enriching pathogen nucleic acids, if present. In some embodiments, the methods described herein comprise enriching host nucleic acids. In some embodiments, the methods described herein comprise enriching host nucleic acids and pathogen nucleic acids, if present, from a sample from the individual.

In some embodiments, enriching nucleic acids, such as host nucleic acids and/or pathogen nucleic acids, if present, comprises subjecting the sample to an oligonucleotide-based affinity matrix. In some embodiments, the oligonucleotide-based affinity matrix is contained in a microfluidic flow cell. In some embodiments, subjecting the sample to the oligonucleotide-based affinity matrix comprises at least 2 cycles, such as at least any of 5 cycles, 10 cycles, 15 cycles, 20 cycles, 25 cycles, 30 cycles, or 35 cycles, of subjecting the sample to the oligonucleotide-based affinity matrix.

In some embodiments, enriching nucleic acids, such as host nucleic acids and pathogen nucleic acids, if present, further comprises eluting captured nucleic acids from the oligonucleotide-based affinity matrix. In some embodiments, eluting captured nucleic acids from the oligonucleotide-based affinity matrix occurs prior to determining the host response profile and the pathogen profile. In some embodiments, nucleic acids captured by the oligonucleotide-based affinity matrix are eluted using an elution buffer, and wherein the elution buffer has a pH of about 3 or less, or the elution buffer has a pH of about 12 or greater.

In some embodiments, the method comprises neutralizing the eluate elution buffer following elution form the oligonucleotide-based affinity matrix.

In some embodiments, the volume of the eluate is less than about 100 μL, such as less than about any of 75 μL, 50 μL, or 25 μL.

In some embodiments, target capture occurs inside a microfluidic flow cell in which oligonucleotides have been synthesized on the inner surfaces. Such process uses photolithography to enable synthesis of oligonucleotides on both sides of the narrow (e.g., less than 50 microns) fluidic channels formed between bonded polymer and glass surfaces. In some embodiments, the oligonucleotides are designed to capture target nucleotides in denaturing conditions. A significant advantage comes from the use of such modified nucleotides as it allows for the binding of specific RNAs under highly denaturing conditions, for example in the presence of 4 M guanidine salts, which break secondary RNA structure and inactivates human ribonucleases. Such approach allows, e.g., the efficient capture of bacterial ribosomal RNAs. Target capture of rRNA is usually inefficient because rRNA has a large amount of secondary and tertiary structure and self-binding is more favorable than hybridization to the capture probe. By running the target capture under denaturing conditions rRNAs are denatured and available to bind to the capture oligonucleotide, however such conditions would normally also prevent hybridization to the capture oligo, the incorporation of modified nucleotides into the capture oligonucleotide allows capture to occur under such typically denaturing conditions. The ability to capture rRNA efficiently dramatically increases the sensitivity of the test because all bacteria contain rRNA and thousands of copies are present per cell, compared to mRNAs which are present at highly variable levels and DNA which is typically present in just a single copy per cell.

The small internal volume (several microliters) of the microfluidic device creates a high local concentration of oligonucleotides probes supporting fast and efficient target capture. This is important because the concentration of bacteria in blood during a typical sepsis infection is frequently lower than a single colony forming unit per ml (ref). This equates to an rRNA copy number of 10¹-10⁴ per ml and thus a large volume of blood must be collected to enable clinically relevant sensitivity. Achieving the oligonucleotide concentration required for rapid solution based capture in a volume of 10 ml or more whilst preventing non-specific binding is prohibitively expensive and challenging and recovery of the captured nucleic acid into a small volume for subsequent detection and identification steps is incredibly challenging. By using the microfluidic device, the oligonucleotide concentration within the flow cell is sufficiently high to provide rapid and efficient target capture whilst the sample is in the flow cell and a large volume of blood can be rapidly circulated through the flow cell multiple times to achieve efficient capture from a high volume sample. This enables capture to be completed within a time frame of 30-90 minutes, allowing an end-end test workflow that can be completed in a clinically meaningful time (i.e. <6 hours). Because the capture probes are bound to a surface, probe-probe nonspecific interactions are prevented even at very high concentrations. Because of the small volume of the flow cell, captured nucleic acids can be eluted into a very small volume (<50 μl) after capture is complete. This allows the specific nucleic acid of interest to be enriched from background nucleic acid but also concentrated from a volume of 5-10 ml or more into a volume of <50 μl.

In some embodiments, for bacterial identification, capture probes target 16S and/or 23S ribosomal RNAs, typically present in bacterial in thousands of copies. Several sequences in ribosomal RNA are conserved in all bacteria and are used for construction of oligonucleotide probes.

In some embodiments, host response is assessed by targeting mRNAs of biomarker genes previously validated to differentiate infection-positive (sepsis) from infection-negative systemic inflammation in patients suspected of sepsis. In some embodiments, the RNA biomarker panel will consist of EACAM4, LAMP1, PLA2G7, and PLAC8 (FDA clearance K163260), and FAIM3.

In some embodiments, the pathogen profile comprises attribute information about a pathogen in the sample from the individual. For example, a panel may includes probes for bacterial mRNAs conferring antimicrobial resistance: carbapenemases, ESBL bacterial, MRSA, and VRE.

In some embodiments, the RNA material retained in a capture device will be eluted using extreme pH conditions (for example about <3 or >12), and immediately neutralized following elution and then used for PCR amplification.

In some aspects, the methods described herein comprise determining a host response profile and a pathogen profile.

In some embodiments, determining the host response profile comprises detecting the enriched host nucleic acids. In some embodiments, detecting the enriched host nucleic acids comprises subjecting the enriched host nucleic acids to an amplification technique. In some embodiments, determining the host response profile comprises detecting host polypeptides.

In some embodiments, determining the pathogen profile comprises detecting, such as detecting the presence, of the enriched pathogen nucleic acids, if present. In some embodiments, determining the pathogen profile comprises detecting, such as detecting the presence, or the enriched pathogen nucleic acids, if present, following subjecting a sample from the individual to an enrichment and/or amplification technique. In some embodiments, determining the pathogen profile comprises not detecting pathogen nucleic acids following subjecting a sample from the individual to an enrichment and/or amplification technique.

In some embodiments, detecting the enriched pathogen nucleic acids comprises subjecting the enriched pathogen nucleic acids to an amplification technique. In some embodiments, detecting the enriched host nucleic acids comprises subjecting the enriched host nucleic acids to an amplification technique. In some embodiments, the amplification technique is a PCR technique. In some embodiments, the PCR technique comprises a reverse transcription PCR (RT-PCR) technique and/or a quantification PCR (qPCR) technique.

In some embodiments, determining the pathogen profile comprises determining the identification of a pathogen. In some embodiments, determining the identification of the pathogen comprises subjecting the amplified pathogen nucleic acids to a nucleic acid sequencing technique. In some embodiments, the nucleic acid sequencing technique is a nanopore-based sequencing technique.

In some embodiments, the methods described herein are designed to enable initial amplification of the entire volume of the eluted material from an enrichment step in a single PCR reaction, such as RT-PCR. In some embodiments, the methods described herein comprise a 2-stage amplification strategy, wherein the initial RT-PCR using multiple primer pairs is conducted for 10-15 cycles using DNA Polymerase enzyme with a strong reverse transcriptase activity, for example Hawk ZO5 DNA Polymerase (Roche Applied Science). In some embodiments, oligonucleotide primers employed in this stage incorporate a specifically designed common sequence element at 5′-end and gene-specific sequence at 3′-end directing reverse transcription and PCR amplification. In some embodiments, the RT-PCR reaction comprises primers amplifying a short (less than 100 bases) sequence in 16S RNA highly conserved in all bacteria, and primers directed to human RNA biomarkers. In some embodiments, at the competition of the first stage, aliquots of the reaction are transferred into separate tubes containing primers and probes toward highly conserved 16S RNA sequence and human biomarker RNAs. In some embodiments, the reaction is amplified for additional 30-40 cycles in a fast real-time PCR cycler (such as MIC cycler from Bio Molecular Systems) completing amplification within 30 minutes. In some embodiments, the absence of 16S bacterial RNA, as determined by the lack of Ct value within expected range, will indicate absence of bacterial infection and will be reported as a negative result. In some embodiments, the presence of highly conserved 16S RNA sequence detected by real-time PCR will suggest potential bacterial infection and will support further interrogation of such sample, e.g., to include additional information in the pathogen profile. In some embodiments, the expression index for human biomarkers will be calculated and compared with the bacterial detection result.

In some embodiments, absence of bacterial detection and low score of biomarker expression index will support ruling out of bacterial etiology of sepsis and a need for antibiotic therapy. In some embodiments, detection of bacterial RNA will trigger further sample evaluation by sequencing to establish pathogen identity. In some embodiments, an additional aliquot of the first-stage RT-PCR reaction will be amplified with a primer pair directed toward common sequence element creating enough DNA material for the nanopore sequencing.

In some embodiments, nanopore sequence will be performed on Minion sequencer from Oxford Nanopore (ONT) using custom -designed workflow for PCR Sequencing Kit (SQK-PSK004). In some embodiments, such flow cells can be utilized for multiple sequencing runs to reduce overall assay cost. In some embodiments, this approach will be supported by encoding sample identity in the common sequence element to make sequencing traces coming from the particular sample easily identifiable. In some embodiments, nanopore sequencing provides sequencing results in real-time. In some embodiments, the sequencing is performed only until enough information is obtained to determining a sequence identity at a certain confidence level.

In some embodiments, results of the sequencing identification will be interpreted in the context of biomarker expression index. In some embodiments, the severity of the sepsis is determined, e.g., from the infection profile, or a component thereof.

In some embodiments, the host response profile provides information regarding the detection of common commensal microorganisms.

In some embodiments, the methods provided herein provide an infection profile comprising clinically relevant information useful for diagnosing the individual. In some embodiments, the host response will be determined by measuring expression levels of biomarker genes and will be presented as a score to estimate likelihood of infection-positive (sepsis) vs infection-negative systemic inflammation in patients suspected of sepsis. In some embodiments, bacterial presence will be assessed by detection of bacterial ribosomal RNA as a biomarker of an active infection.

In some embodiments, high score of host response and positive detection of bacterial presence will definitely diagnose sepsis and will require immediate antibiotic treatment.

In some embodiments, low score of host response and absence of bacterial detection will definitely rule out bacterial infection and will support withdrawal of antibiotic treatment.

In some embodiments, high score of host response and absence of bacterial detection will indicate potential diagnosis of sepsis of non-bacterial etiology and potential withdrawal of antibiotic treatment.

In some embodiments, low score of host response and positive detection of bacterial presence will require further testing to establish identification of the detected bacterial. In some embodiments, bacterial contamination during blood collection by several commensal microorganisms may be differentiated from real opportunistic infection by the same microorganisms based on the host response score. In some embodiments, low host response will be expected in the case of transient bacteremia caused by commensal bacteria typically present in human microbiota. In some embodiments, additional testing may be required to establish need for antibiotic therapy in this case.

In some embodiments, the methods provided herein further comprise processing the sample prior to the step of enriching.

In some embodiments, the methods described herein are completed in 24 hours or less, such as any of 18 hours or less, 12 hours or less, 8 hours or less, or 6 hours or less.

In some embodiments, the sample is any biological sample from an individual. In some embodiments, the sample is a sterile sample. In some embodiments, the sample is a blood sample. In some embodiments, the sample is a whole blood sample. In some embodiments, the sample is a serum sample. In some embodiments, the sample is a plasma sample. In some embodiments, the sample is seminal fluid. In some embodiments, the sample is CNS fluid.

In some embodiments, the sample volume is at least about 1 mL. In some embodiments, the sample volume is about 1 mL to about 15 mL, such as about 3 mL to about 10 mL. In some embodiments, the sample volume is less than about 15 mL, such as less than about 12 mL or less than about 10 mL.

In some embodiments, the sample, such as a blood sample, is collected into denaturing buffer in a container, such as a vacutainer. In some embodiments, the sample is mixed with a denaturing buffer. In some embodiments, the denaturing buffer is in the container prior to addition of the sample. In some embodiments, the denaturing buffer lyses cells, such as bacterial cells and human cells, and stabilizes nucleic acids.

In some embodiments, the host nucleic acids are RNA. In some embodiments, the host nucleic acids are mRNA.

In some embodiments, the pathogen nucleic acids comprise nucleic acids from one or more pathogens. In some embodiments, the pathogen nucleic acids comprise nucleic acids from a plurality of pathogens, wherein the plurality of pathogens comprises two or more species of pathogens. In some embodiments, the pathogen nucleic acids are RNA. In some embodiments, the pathogen nucleic acids are rRNA.

In some embodiments, the sample is a whole blood sample. In some embodiments, the volume of the whole blood sample is about 1 ml to about 15 ml.

In some embodiments, the methods described herein, such as determining an infection profile, comprise: (a) capturing host nucleic acids and pathogen nucleic acids, if present, from a sample from an individual; (b) eluting the captured nucleic acids; (c) amplifying the captured nucleic acids; (d) detecting host nucleic acids and pathogen nucleic acids, if present, and (e) determining a host response profile based on the detection of host nucleic acids and a pathogen profile based on the detection (or lack thereof) of pathogen nucleic acids. In some embodiments, the host response profile is indicative of an infection-positive status, e.g., indicate a likelihood of an infection-positive status. In some embodiments, the host response profile is indicative of an infection-negative status, e.g., indicate a low likelihood of an infection-positive status. In some embodiments, the pathogen profile is indicative of a bacterial presence in the individual. In some embodiments, the pathogen profile is indicative of a non-bacterial presence in the individual. In some embodiments, the pathogen profile is indicative of no bacterial presence in the sample from the individual. In some embodiments, wherein bacterial presence is detected, the methods described herein further comprise sequencing enriched pathogen nucleic acids, e.g., to identify the species of the pathogen and/or an attribute of the pathogen.

In some embodiments, the methods described herein, such as determining an infection profile, comprise: (a) capturing host RNA, such as mRNA, and pathogen RNA, such as rRNA, if present, from a sample from an individual; (b) eluting the captured host RNA and pathogen RNA, if present; (c) amplifying the captured host RNA and pathogen RNA, if present; (d) detecting host RNA and pathogen RNA, if present, and (e) determining a host response profile based on the detection of host RNA and a pathogen profile based on the detection (or lack thereof) of pathogen RNA. In some embodiments, the host response profile is indicative of an infection-positive status, e.g., indicate a likelihood of an infection-positive status. In some embodiments, the host response profile is indicative of an infection-negative status, e.g., indicate a low likelihood of an infection-positive status. In some embodiments, the pathogen profile is indicative of a bacterial presence in the individual. In some embodiments, the pathogen profile is indicative of a non-bacterial presence in the individual. In some embodiments, the pathogen profile is indicative of no bacterial presence in the sample from the individual. In some embodiments, wherein bacterial presence is detected, the methods described herein further comprise sequencing enriched pathogen nucleic acids, e.g., to identify the species of the pathogen and/or an attribute of the pathogen.

In some embodiments, the methods described herein, such as determining an infection profile, comprise: (a) capturing host nucleic acids and pathogen nucleic acids, if present, from a sample from an individual; (b) eluting the captured nucleic acids; (c) amplifying the captured nucleic acids; (d) detecting host nucleic acids and pathogen nucleic acids, if present; (e) sequencing pathogen nucleic acids, if present; and (f) determining a host response profile based on the detection of host nucleic acids and a pathogen profile based on the detection (or lack thereof) of pathogen nucleic acids. In some embodiments, the host response profile is indicative of an infection-positive status, e.g., indicate a likelihood of an infection-positive status. In some embodiments, the host response profile is indicative of an infection-negative status, e.g., indicate a low likelihood of an infection-positive status. In some embodiments, the pathogen profile is indicative of a bacterial presence in the individual. In some embodiments, the pathogen profile is indicative of a non-bacterial presence in the individual. In some embodiments, the pathogen profile is indicative of no bacterial presence in the sample from the individual. In some embodiments, the sequencing information is used to identify the species of the pathogen and/or an attribute of the pathogen.

In some embodiments, provided herein is a method of diagnosing sepsis in an individual, the method comprising determining an infection profile of the individual according to a method described herein.

In some embodiments, provided herein is a method of analyzing a sample from an individual, the method comprising determining an infection profile of the individual according to a method described herein.

In some embodiments, provided herein is a method of differentiating a cause of an infection or infection response in an individual, the method comprising determining an infection profile of the individual according to a method described herein.

In some embodiments, provided herein is a method of diagnosing a condition in an individual, the method comprising determining an infection profile of the individual according to a method described herein.

In some embodiments, provided herein is a method of determining a course of treatment for a condition in an individual, the method comprising determining an infection profile of the individual according to a method described herein, wherein the course of treatment is based on the infection profile of the individual. In some embodiments, the course of treatment comprises selection and administration of an anti-microbial agent. In some embodiments, the course of treatment comprises stopping administration of an anti-microbial agent.

In some embodiments, provided herein is a method of determining the likelihood an individual has sepsis, the method comprising determining an infection profile of the individual according to a method described herein.

In some embodiments, provided herein is a method of determining the severity of sepsis in an individual, the method comprising determining an infection profile of the individual according to a method described herein.

In some embodiments, provided herein is a method of monitoring sepsis in an individual, the method comprising determining an infection profile of the individual according to a method described herein.

In some embodiments, provided herein is a method of differentiating causes of systemic inflammatory response in an individual, the method comprising determining an infection profile of the individual according to a method described herein.

In some embodiments, provided herein is a method of diagnosing an infectious disease in a patient potentially infected with a pathogenic microorganism, wherein the diagnostic result is based on a host response profile and detection of nucleic acids derived from a pathogenic microorganism in the individual, the method comprising: (a) enriching host nucleic acids and pathogen nucleic acids, if present, from a sample from the individual; (b) determining the host response profile and presence or absence of nucleic acids derived from pathogenic microorganisms; and (c) assessing likelihood of the infectious disease potentially caused by pathogenic microorganism based on interpretation of the host response profile and detection of nucleic acids derived from the pathogenic microorganism. In some embodiments, the host response profile is indicative of a pathogen infection in the individual. In some embodiments, the host response profile is indicative of an infection-negative response in the individual. In some embodiments, the detection of nucleic acids derived from pathogenic microorganism is indicative of the presence of a pathogen, or portion thereof, in the sample from the individual. In some embodiments, detection of nucleic acids derived from pathogenic microorganism comprises detection of nucleic acids encoding 16S ribosomal RNA and/or 23S ribosomal RNA. In some embodiments, the detection of nucleic acids derived from the pathogenic microorganism comprises the identification of the pathogen. In some embodiments, the pathogen profile comprises an attribute of the pathogen. In some embodiments, the attribute of the pathogen is anti-microbial resistance. In some embodiments, enriching host nucleic acids and pathogen nucleic acids, if present, comprises subjecting the sample to an oligonucleotide-based affinity matrix. In some embodiments, the oligonucleotide-based affinity matrix is contained in a microfluidic flow cell. In some embodiments, determining the host response profile comprises detecting the enriched host nucleic acids. In some embodiments, determining presence of nucleic acids derived from a pathogenic microorganism comprises determining the identification of a pathogen.

In some embodiments, provided herein is a method of differentiating a disease-causing infection in a patient potentially infected with a pathogenic microorganism from a non-disease causing infection or specimen contamination by a microorganism, wherein the diagnostic result is based on a host response profile and on detection of nucleic acids derived from a pathogenic microorganism of the individual, the method comprising: (a) enriching host nucleic acids and pathogen nucleic acids, if present, from a sample from the individual; (b) determining the host response profile and presence or absence of nucleic acids derived from pathogenic microorganisms; and (c) assessing likelihood of infectious disease potentially caused by pathogenic microorganism based on interpretation of the host response profile and detection of nucleic acids derived from the pathogenic microorganism.

The disclosure provided herein is further illustrated by the following examples, which are not to be construed as limiting the disclosure in scope or spirit to the specific procedures described in them.

### EXAMPLES

### Example 1

This example demonstrates an exemplary workflow of the methods described herein.

The test workflow will comprise binding of targeted bacterial and human RNAs on oligonucleotide-based affinity matrix, amplification and detection by qPCR, and rapid identification using nanopore sequencing. This approach is taking advantage of conserved sequences present in bacterial RNA to capture specific targets and to drive PCR amplification.

Whole blood will be collected into denaturing buffer in a vacutainer. Blood volume required for the desired test sensitivity is estimated to be at least 3 ml but ideally up to 10 ml of blood will be collected and the test will be capable of processing at least 12 ml of blood. The denaturing buffer in the vacutainer will serve to lyse both bacterial and human cells that are present as well as stabilizing nucleic acids in the sample by denaturing nuclease enzymes. This step is included as RNA is a highly transient substance and can be rapidly broken down within cells and, upon lysis, many cells release high levels of nuclease enzymes. In order to enable detection of high levels of RNA, cells must be quickly lysed and the RNA preserved. This lysis process also serves to release bacterial cells and nucleic acids present inside human white blood cells that have engulfed the bacteria. Without lysis, the typical process of degradation inside the white blood cell would proceed for some time during transport of the specimen, thereby reducing the amount of detectable RNA in the specimen by the time it is tested.

The lysed blood sample is then used for direct affinity-based purification of targeted bacterial RNA and a set of human mRNAs within a microfluidic device. Retained nucleic acids will be eluted and amplified in two stage RT-PCR amplification. Amplified DNA is detected and quantified by qPCR and identified using rapid nanopore-based sequencing. This novel combination of technologies provides a hypothesis-free approach to bacterial detection, creating a unique capability to rapidly detect any bacterial pathogen and to use this information in a context of sepsis severity.

Target capture will occur inside a microfluidic flow cell in which oligonucleotides have been synthesized on the inner surfaces. The process will use photolithography to enable synthesis of oligonucleotides on both sides of the narrow (less than 50 microns) fluidic channels formed between bonded polymer and glass surfaces. Capture oligonucleotides will be designed to bind target RNA under denaturing conditions. By running the target capture under denaturing conditions rRNAs will be denatured and available to bind to the capture oligonucleotide. Such conditions will normally also prevent hybridization to the capture oligonucleotide, thus the incorporation of such nucleotides into the capture oligonucleotide will allow capture to occur under such conditions. This approach to capturing rRNA efficiently dramatically increases the sensitivity of the test because all bacteria contain rRNA and thousands of copies are present per cell, compared to mRNAs which are present at highly variable levels and DNA which is typically present in just a single copy per cell.

The small internal volume (several microliters) of the microfluidic device will create a high local concentration of oligonucleotides probes supporting fast and efficient target capture. This is important because the concentration of bacteria in blood during a typical sepsis infection is frequently lower than a single colony forming unit per ml. This equates to an rRNA copy number of 10¹-10⁴ per ml and so a large volume of blood must be collected to enable clinically relevant sensitivity. Achieving the oligonucleotide concentration required for rapid solution based capture in a volume of 10 ml or more whilst preventing non-specific binding is prohibitively expensive and challenging and recovery of the captured nucleic acid into a small volume for subsequent detection and identification steps is incredibly challenging. By using the present microfluidic device, the oligonucleotide concentration within the flowcell is sufficiently high to provide rapid and efficient target capture whilst the sample is in the flowcell and a large volume of blood can be rapidly circulated through the flow cell multiple times to achieve efficient capture from a high volume sample. This enables capture to be completed within a time frame of 30-90 minutes, allowing an end-end test workflow that can be completed in a clinically meaningful time (e.g., <6 hours). The capture probes are bound to a surface and probe-probe nonspecific interactions are prevented even at very high concentrations.

The small volume of the flow cell will allow for the captured nucleic acids to be eluted into a very small volume (<50 μl) after capture is complete. This allows the specific nucleic acid of interest to be enriched from background nucleic acid but also concentrated from a volume of 5-10 ml or more into a volume of <50 μl.

Capture probes targeting 16S and 23S ribosomal RNAs, which are typically present in bacterial in thousands of copies, will allow for bacterial identification. Several sequences in ribosomal RNA, are conserved in all bacteria.

Host response will be addressed by targeting mRNAs of biomarker genes validated to differentiate infection-positive (sepsis) from infection-negative systemic inflammation in patients suspected of sepsis. For example, the RNA biomarker panel will consist of one or more of EACAM4, LAMP1, PLA2G7, and PLAC8 (FDA clearance K163260), and FAIM3.

In addition, our panel includes probes for bacterial mRNAs conferring antimicrobial resistance: carbapenemases, ESBL bacterial, MRSA and VRE. The RNA material retained in a capture device will be eluted using extreme pH conditions (for example <3 or >12), immediately neutralized and used for RT-PCR amplification.

The fluidic design of capture device enables initial amplification of the entire volume of the eluted material in a single RT-PCR reaction. The described method will use a 2-stage amplification strategy, wherein the initial RT-PCR using multiple primer pairs is conducted for 10-15 cycles using DNA Polymerase enzyme with a strong reverse transcriptase activity, for example Hawk ZO5 DNA Polymerase (Roche Applied Science). Oligonucleotide primers employed in this stage incorporate a specifically designed common sequence element at 5′-end and gene-specific sequence at 3′-end directing reverse transcription and PCR amplification. In addition, the RT-PCR reaction will contain primers amplifying a short (less than 100 bases) sequence in 16S RNA highly conserved in all bacteria, and primers directed to human RNA biomarkers. At the competition of the first stage, aliquots of the reaction will be transferred into separate tubes containing primers and probes toward highly conserved 16S RNA sequence and human biomarker RNAs. The reaction is amplified for additional 30-40 cycles in a fast real-time PCR cycler (such as MIC cycler from Bio Molecular Systems) completing amplification within 30 minutes. The absence of 16S bacterial RNA, as determined by the lack of Ct value within expected range, will indicate absence of bacterial infection and will be reported as a negative result. The presence of highly conserved 16S RNA sequence detected by real-time PCR suggests potential bacterial infection and will support further interrogation of such sample. The expression index for human biomarkers will be calculated and compared with the bacterial detection result.

Absence of bacterial detection and low score of biomarker expression index will support ruling out of bacterial etiology of sepsis and a need for antibiotic therapy. The detection of bacterial RNA will trigger further sample evaluation by sequencing to establish pathogen identity.

An additional aliquot of the first-stage RT-PCR reaction will be amplified with a primer pair directed toward common sequence element creating enough DNA material for the nanopore sequencing.

The nanopore sequence will be performed on Minion sequencer from Oxford Nanopore (ONT) using a workflow for PCR Sequencing Kit (SQK-PSK004). This method is designed to re-use Minion flow cells for multiple sequencing runs to reduce overall assay cost. This approach is supported by encoding sample identity in the common sequence element to make sequencing traces coming from the particular sample easily identifiable. The key advantage of nanopore sequencing technology is its ability to generate sequencing results in real-time. Our preliminary data indicated that 15 minutes nanopore run is enough to perform bacterial ID with more than 99.5% accuracy. Our data also indicated that low accuracy, which is considered to be a common problem of nanopore sequencing, can be overcome by sequencing amplified DNA fragments and applying custom consensus assembly prior to sequence mapping to the database. A practical example of high accuracy of the suggested approach was demonstrated by correct identification of ribosomal RNA sequences of Neisseria Gonorrhoeae and Neisseria Meningitidis, which share more than 98.5% nucleotide identity.

Results of the sequencing ID will be interpreted in the context of a biomarker expression index. Severity of sepsis will be assessed based on the biomarker expression index. In addition, the host response will allow for address detection of common commensal microorganisms, which could be a result of specimen contamination or a real opportunistic infection. The presence or absence of most common antibiotic resistance genes will help selecting the most appropriate antibiotic therapy on the day of admission.

An initial assessment for bacterial presence (less than 1.5 hours) will be conducted. This will be followed by identification of the bacterial pathogen and presence of several most significant markers of antibiotic resistance (about 1.5-2 hours).

What is claimed is:

1. A method of determining an infection profile of an individual,
wherein the infection profile is based on a host response profile and a pathogen profile of the individual,
the method comprising:
(a) enriching host nucleic acids and pathogen nucleic acids, if present, from a sample from the individual; and
(b) determining the host response profile and the pathogen profile to determine the infection profile of the individual.

2. The method of claim 1, wherein the host response profile is indicative of a pathogen infection in the individual.

3. The method of claim 2, wherein the pathogen infection is a bacterial infection.

4. The method of claim 2, wherein the pathogen infection is a non-bacterial infection.

5. The method of claim 1, wherein the host response profile is indicative of an infection-negative response in the individual.

6. The method of claim 5, wherein the infection-negative response is infection-negative systemic inflammation.

7. The method of any one of claims 1-6, wherein the host response profile is based on host nucleic acid levels of one or more host response biomarkers.

8. The method of claim 7, wherein the one or more host response biomarkers are selected from any of EACAM4, LAMP1, PLA2G7, PLAC8, or FAIM3.

9. The method of claim 7 or 8, wherein the host response profile comprises a host response score based on the one or more host response biomarkers.

10. The method of any one of claims 1-9, wherein the pathogen profile is indicative of the presence of a pathogen, or portion thereof, in the sample from the individual.

11. The method of any one of claims 1-10, wherein the pathogen profile is based on the presence of pathogen nucleic acids.

12. The method of claim 11, wherein the pathogen nucleic acids comprise 16S and/or 23S nucleic acids.

13. The method of claim 12, wherein the 16S and/or 23S nucleic acids comprise conserved sequences of ribosomal RNA (rRNA) of 16S and/or 23S.

14. The method of any one of claims 1-13, wherein the pathogen profile comprises the identification of the pathogen.

15. The method of claim 14, wherein the pathogen profile comprises an attribute of the pathogen.

16. The method of claim 15, wherein the attribute of the pathogen is anti-microbial resistance.

17. The method of claim 16, wherein anti-microbial resistance of the pathogen is based on one or more anti-microbial resistance biomarkers.

18. The method of claim 17, wherein the one or more anti-microbial resistance biomarkers are indicative of presence of a carbapenemase, presence of an extended spectrum beta-lactamase, methicillin resistance, or vancomycin resistance.

19. The method of any one of claims 1-18, wherein the infection profile comprises:
(i) a host response profile indicative of a pathogen infection in the individual; and
(ii) a pathogen profile indicative of the presence of a bacterial pathogen in the individual.

20. The method of any one of claims 1-18, wherein the infection profile comprises:
(i) a host response profile indicative a pathogen infection in the individual; and
(ii) a pathogen profile indicative of the presence of a non-bacterial pathogen in the individual.

21. The method of claim 20, wherein the non-bacterial pathogen is a fungus, parasite, or virus.

22. The method of any one of claims 1-18, wherein the infection profile comprises:
(i) a host response profile indicative of an infection-negative response in the individual; and
(ii) a pathogen profile indicative of the presence of a bacterial pathogen in the individual.

23. The method of any one of claims 1-18, wherein the infection profile comprises:
(i) a host response profile indicative of an infection-negative response in the individual; and
(ii) a pathogen profile indicative of the presence of a non-bacterial pathogen in the individual.

24. The method of claim 23, wherein the non-bacterial pathogen is a fungus, parasite or virus.

25. The method of any one of claims 1-24, wherein enriching host nucleic acids and pathogen nucleic acids, if present, comprises subjecting the sample to an oligonucleotide-based affinity matrix.

26. The method of claim 25, wherein the oligonucleotide-based affinity matrix is contained in a microfluidic flow cell.

27. The method of claim 25 or 26, wherein subjecting the sample to the oligonucleotide-based affinity matrix comprises at least 2 cycles of subjecting the sample to the oligonucleotide-based affinity matrix.

28. The method of any one of claims 25-27, wherein enriching host nucleic acids and pathogen nucleic acids, if present, further comprises eluting captured nucleic acids from the oligonucleotide-based affinity matrix.

29. The method of claim 28, wherein eluting captured nucleic acids from the oligonucleotide-based affinity matrix occurs prior to determining the host response profile and the pathogen profile.

30. The method of claim 28 or 29, wherein nucleic acids are eluted using an elution buffer, and wherein the elution buffer has a pH of about 3 or less, or the elution buffer has a pH of about 12 or greater.

31. The method of claim 30, wherein the pH of the elution buffer is neutralized following elution.

32. The method of any one of claims 28-31, wherein the volume of the eluate is less than about 100 μL.

33. The method of any one of claims 1-32, wherein determining the host response profile comprises detecting the enriched host nucleic acids.

34. The method of claim 33, wherein detecting the enriched host nucleic acids comprises subjecting the enriched host nucleic acids to an amplification technique.

35. The method of any one of claims 1-34, wherein determining the pathogen profile comprises detecting the enriched pathogen nucleic acids.

36. The method of claim 35, wherein detecting the enriched pathogen nucleic acids comprises subjecting the enriched pathogen nucleic acids to an amplification technique.

37. The method of claim 34 or 35, wherein the amplification technique is a PCR technique.

38. The method of claim 37, wherein the PCR technique comprises a reverse transcription PCR (RT-PCR) technique and/or a quantification PCR (qPCR) technique.

39. The method of any one of claims 35-38, wherein determining the pathogen profile comprises determining the identification of a pathogen.

40. The method of claim 39, wherein determining the identification of the pathogen comprises subjecting the amplified pathogen nucleic acids to a nucleic acid sequencing technique.

41. The method of claim 40, wherein the nucleic acid sequencing technique is a nanopore-based sequencing technique.

42. The method of any one of claims 1-41, wherein the host nucleic acids are RNA.

43. The method of claim 42, wherein the host nucleic acids are mRNA.

44. The method of any one of claims 1-43, wherein the pathogen nucleic acids comprise nucleic acids from one or more pathogens.

45. The method of any one of claims 1-43, wherein the pathogen nucleic acids comprise nucleic acids from a plurality of pathogens, wherein the plurality of pathogens comprises two or more species of pathogens.

46. The method of any one of claims 1-45, wherein the pathogen nucleic acids are RNA.

47. The method of claim 46, wherein the pathogen nucleic acids are rRNA.

48. The method of any one of claims 1-47, wherein the sample is a whole blood sample.

49. The method of claim 48, wherein the volume of the whole blood sample is about 1 ml to about 15 ml.

50. The method of any one of claims 1-49, further comprising processing the sample prior to the step of enriching.

51. The method of any one of claims 1-50, wherein the method is completed in 24 hours or less.

52. A method of diagnosing sepsis in an individual comprising performing the method of any one of claims 1-51.

53. A method of diagnosing an infectious disease in a patient potentially infected with a pathogenic microorganism,
wherein the diagnostic result is based on a host response profile and detection of nucleic acids derived from a pathogenic microorganism in the individual,
the method comprising:
(a) enriching host nucleic acids and pathogen nucleic acids, if present, from a sample from the individual;
(b) determining the host response profile and presence or absence of nucleic acids derived from pathogenic microorganisms; and
(c) assessing likelihood of the infectious disease potentially caused by pathogenic microorganism based on interpretation of the host response profile and detection of nucleic acids derived from the pathogenic microorganism.

54. The method of claim 53, wherein the host response profile is indicative of a pathogen infection in the individual.

55. The method of claim 53, wherein the host response profile is indicative of an infection-negative response in the individual.

56. The method of any one of claims 53-55, wherein the detection of nucleic acids derived from pathogenic microorganism is indicative of the presence of a pathogen, or portion thereof, in the sample from the individual.

57. The method of claim 56, wherein detection of nucleic acids derived from pathogenic microorganism comprises detection of nucleic acids encoding 16S ribosomal RNA and/or 23S ribosomal RNA.

58. The method of any one of claims 53-57, wherein the detection of nucleic acids derived from the pathogenic microorganism comprises the identification of the pathogen.

59. The method of claim 58, wherein the pathogen profile comprises an attribute of the pathogen.

60. The method of claim 59, wherein the attribute of the pathogen is anti-microbial resistance.

61. The method of any one of claims 53-60, wherein enriching host nucleic acids and pathogen nucleic acids, if present, comprises subjecting the sample to an oligonucleotide-based affinity matrix.

62. The method of claim 61, wherein the oligonucleotide-based affinity matrix is contained in a microfluidic flow cell.

63. The method of any one of claims 53-62, wherein determining the host response profile comprises detecting the enriched host nucleic acids.

64. The method of any one of claims 53-63, wherein the determining presence of nucleic acids derived from a pathogenic microorganism comprises determining the identification of a pathogen.

65. A method of differentiating a disease-causing infection in a patient potentially infected with a pathogenic microorganism from a non-disease causing infection or specimen contamination by a microorganism,
wherein the diagnostic result is based on a host response profile and on detection of nucleic acids derived from a pathogenic microorganism of the individual,
the method comprising:
(a) enriching host nucleic acids and pathogen nucleic acids, if present, from a sample from the individual;
(b) determining the host response profile and presence or absence of nucleic acids derived from pathogenic microorganisms; and
(c) assessing likelihood of infectious disease potentially caused by pathogenic microorganism based on interpretation of the host response profile and detection of nucleic acids derived from the pathogenic microorganism.

66. A method of diagnosing an infectious disease in a patient potentially infected with a pathogenic microorganism,
wherein the diagnostic result is based on a measurement of host biomarker and detection of nucleic acids derived from a pathogenic microorganism in the individual,
the method comprising:
(a) enriching pathogen nucleic acids, if present, from a sample from the individual;
(b) performing measurement of host biomarker and presence or absence of nucleic acids derived from pathogenic microorganisms;
(c) assessing likelihood of the infectious disease potentially caused by pathogenic microorganism based on interpretation of the measurement of host biomarker and detection of nucleic acids derived from the pathogenic microorganism; and
(d) further assessing likelihood of the presence of pathogen-derived nucleic acids comprising genes responsible or associated with antimicrobial resistance.

67. The method of claim 66, wherein results of the test are provided within less than 8 hours.
